SEQUENCE LISTING (16 sequences)

SEQ ID NO: 1 - DNA, 30 bp, Artificial Sequence
  Synthetic
gnggngcacaagnaacgganannacaanca

SEQ ID NO: 2 - DNA, 27 bp, Artificial Sequence
  Synthetic
aaacaagcaaacgcacaancagaagct

SEQ ID NO: 3 - DNA, 33 bp, Artificial Sequence
  Synthetic
acacannacnganggnnnngannncnnaggcnt

SEQ ID NO: 4 - DNA, 33 bp, Artificial Sequence
  Synthetic
gngnaaaaggnaaaaaanggagnccgannaagg

SEQ ID NO: 5 - DNA, 26 bp, Artificial Sequence
  Synthetic
ctcctgctgctttcgcatggttaatc

SEQ ID NO: 6 - DNA, 31 bp, Artificial Sequence
  Synthetic
tctgtaccataactagcatttgtgctttgaa

SEQ ID NO: 7 - DNA, 31 bp, Artificial Sequence
  Synthetic
tgacactttggttggatggtttaatgaagag

SEQ ID NO: 8 - DNA, 33 bp, Artificial Sequence
  Synthetic
tggagattcttcaacgcaatataccctactaaa

SEQ ID NO: 9 - DNA, 76 bp, Artificial Sequence
  Synthetic
nnnnnnnnnnnnnnnnnnnnnnnnnnnnnnnnnnnnnnnnynnnnnnnnnnnnnnnnnnnnnnnnnnnnnnnnnnn

SEQ ID NO: 10 - DNA, 76 bp, Artificial Sequence
  Synthetic
nnnnnnnnnnnnnnnnnnnnnnnnnnnnnnnnnnnrnnnnnnnnnnnnnnnnnnnnnnnnnnnnnnnnnnnnnnnn

SEQ ID NO: 11 - DNA, 48 bp, Artificial Sequence
  Synthetic
nnnnnnnnnnnnynnnnnnnnnnnnnnnnnnnnnnnnnnnnnnnnnnn

SEQ ID NO: 12 - DNA, 49 bp, Artificial Sequence
  Synthetic
nnnnnnnnrnnnnnnnnnnnnnnnnnnnnnnnnnnnnnnnnnnnnnnnn

SEQ ID NO: 13 - DNA, 47 bp, Artificial Sequence
  Synthetic
nnnnnnnnnnnynnnnnnnnnnnnnnnnnnnnnnnnnnnnnnnnnnn

SEQ ID NO: 14 - DNA, 35 bp, Artificial Sequence
  Synthetic
nnnnnnnnnnnnnnnnnnnnnnnnnnnnnnnnnnn

SEQ ID NO: 15 - DNA, 49 bp, Artificial Sequence; identical to SEQ ID NO: 12
  Synthetic

SEQ ID NO: 16 - DNA, 40 bp, Artificial Sequence
  Synthetic
nnnnnnnnnnnnnnnnnnnnnnnnnnnnnnnnnnnnnnnn

(19) United States
(12) Patent Application Publication
(10) Pub. No.: US 2016/0122810 A1
(43) Pub. Date: May 5, 2016

(54) SYSTEMS AND METHODS FOR NUCLEIC ACID CAPTURE

(71) Applicant: Ibis Biosciences, Inc., Carlsbad, CA (US)

(72) Inventors: Thomas N. Chiesl, Carlsbad, CA (US); Steven G. Haupt, Carlsbad, CA (US); Lendell L. Cummins, Carlsbad, CA (US); Christopher D. Crowder, Carlsbad, CA (US)

(21) Appl. No.: 14/920,666

(22) Filed: Oct. 22, 2015

Related U.S. Application Data
(60) Provisional application No. 62/067,256, filed on Oct. 22, 2014.

(51) Int. Cl. C12Q 1/68

(52) U.S. Cl. CPC C12Q 1/6853; C12Q 1/6837

(57) ABSTRACT

Provided herein are methods for improving the detection sensitivity of amplification reaction products. In particular, provided herein are methods of improving sensitivity of detection of amplification products by introducing modified and degradable nucleotides into amplification primers.

### CROSS-REFERENCE TO RELATED APPLICATIONS

The present Application claims priority to U.S. Provisional Application Ser. No. 62/067,256 filed Oct. 22, 2014, the entirety of which is incorporated by reference herein.

### FIELD OF THE DISCLOSURE

Provided herein are methods for improving the detection sensitivity of amplification reaction products. In particular, provided herein are methods of improving sensitivity of detection of amplification products by introducing modified and degradable nucleotides into amplification primers.

### BACKGROUND

Nucleic acid amplification reactions are crucial for many research, medical, and industrial applications. Such reactions are used in clinical and biological research, detection and monitoring of infectious diseases, detection of mutations, detection of cancer markers, environmental monitoring, genetic identification, detection of pathogens in biodefense applications, and the like, e.g. Schweitzer et al., Current Opinion in Biotechnology, 12: 21-27 (2001); Koch, Nature Reviews Drug Discovery, 3: 749-761 (2004). In particular, polymerase chain reactions (PCRs) have found applications in all of these areas, including applications for viral and bacterial detection, viral load monitoring, detection of rare and/or difficult-to-culture pathogens, rapid detection of bio-terror threats, detection of minimal residual disease in cancer patients, food pathogen testing, blood supply screening, and the like, e.g. Mackay, Clin. Microbiol. Infect., 10: 190-212 (2004); Bernard et al., Clinical Chemistry, 48: 1178-1185 (2002). In regard to PCR, key reasons for such widespread use are its speed and ease of use (typically performed within a few hours using standardized kits and relatively simple and low cost instruments), its sensitivity (often a few tens of copies of a target sequence in a sample can be detected), and its robustness (poor quality samples or preserved samples, such as forensic samples or fixed tissue samples are readily analyzed), Strachan and Read, Human Molecular Genetics 2 (John Wiley & Sons, New York, 1999).

Despite the advances in nucleic acid amplification techniques that are reflected in such widespread applications, there is still a need for further improvements in speed and sensitivity, particularly in such areas as infectious disease detection, minimum residual disease detection, bio-defense applications, and the like.

### SUMMARY OF THE DISCLOSURE

Provided herein are methods for improving the detection sensitivity of amplification reaction products. In particular, provided herein are methods of improving sensitivity of detection of amplification products by introducing modified and degradable nucleotides into amplification primers.

For example, in some embodiments, the present disclosure provides a system, comprising one or more of: a) a plurality of amplification primer pairs specific for a target nucleic acid (e.g., RNA or DNA), wherein at least one thymidine nucleotide in one of the primers of each pair is replaced with 2′-deoxyuridine and the other primer of each of the pairs comprises a detectable label; b) an enzyme that specifically degrades uracil (e.g., Uracil-DNA glycosylase (UDG) enzyme; c) a solid support comprising a plurality of nucleic acids complementary to the target nucleic acids; and d) a detection device. In some embodiments, the detection device is an optical, colorimetric, chemiluminescent, radio isotopic, chemical change (e.g. pH), magnetic detection device (e.g., Hall sensor), electrical (e.g., nanowire detections that measure a change in resistance or voltage measured from the presence of hybridized DNA), or electrochemical detection device. In some embodiments, the solid support is a microarray. In some embodiments, the nucleic acids complementary to the target nucleic acids (e.g., capture probes) extend into the amplification products. In some embodiments, the nucleic acids complementary to the target nucleic acids comprise DNA, peptide-nucleic acids, modified nucleic acids (e.g., locked nucleic acids, bridged nucleic acids, or thiophosphoramidites), or a detectable label (e.g., fluorescent labels, quenchers, etc., present as part of a modified base or attached to a nucleic acid). In some embodiments, the primer pairs are selected from PCR primers, isothermal amplification primers, or whole genome amplification primers. In some embodiments, at least two, three, four, five, or all of the thymidine nucleotide in one of the primers of each pair is replaced with 2′-deoxyuridine. In some embodiments, the amplification is PCR, reverse transcriptase PCR (RT-PCR), whole genome amplification, isothermal amplification, or rolling circle replication.

Further embodiments provide a method, comprising: a) contacting a sample comprising a target nucleic acid with a plurality of amplification primer pairs specific for a target nucleic acid, wherein at least one thymidine nucleotide in one or the primers of each pair is replaced with 2′-deoxyuridine and the other primer of each of the pairs comprises a detectable label; b) performing an amplification reaction to amplify the target nucleic acid; c) treating the amplification reaction with a UDG enzyme; and d) capturing the target nucleic acid on a solid support comprising a plurality of nucleic acids complementary to the target nucleic acids.

Additional embodiments comprise a reaction mixture comprising at least one complex of a target nucleic acid and one or more amplification primers, wherein at least one (e.g., two or more, three or more, or all) thymidine nucleotide in one or more of the primers is replaced with 2′-deoxyuridine. In some embodiments, one of the primers comprises a detectable label or quencher. In some embodiments, the primers are PCR primers, isothermal amplification primers, sequencing primers, or whole genome amplification primers. In some embodiments, reaction mixtures comprise one or more extended primers (e.g., comprising one or more 2′-deoxyuridine nucleotides), double stranded amplicons (e.g., not comprising 2′-deoxyuridine nucleotides), enzymes (e.g., nucleic acid polymerases, UDG enzymes, etc.), nucleotides, buffers, etc. In some embodiments, reaction mixtures comprise one or more sequencing primers comprising at least one 2′-deoxyuridine hybridized to an amplicon having primer sequences and lacking 2′-deoxyuridine. In some embodiments, reaction mixtures comprise fragmented primers that once had 2′-deoxyuridine (e.g., that have been treated with a UDG enzyme) and an amplicon. In some embodiments, reaction mixtures comprise an extended 2′-deoxyuridine containing primer hybridized to a nucleic acid comprising a target sequence or an amplicon. In some embodiments, one or more components of the reaction mixture or a target capture nucleic acid are bound to a solid support (e.g., microarray, bead, particle, etc.).

Additional embodiments are described herein.

### DESCRIPTION OF THE FIGURES

FIG. 1 shows forward primers with barcodes sequences attached with linkers.

FIG. 2 shows a demonstration of how unlabeled or unincorporated forward product with a bar code can compete for binding to array spot reducing signal.

FIG. 3 shows a bar chart of average pixel intensity showing how increased concentrations of unlabeled product can compete for binding to the array and reduce signal intensity. X-axis labels denote ratio of labeled to unabled product.

FIG. 4 shows a schematic of a method of embodiments of the present disclosure to avoid binding competition at the array spot.

FIG. 5 shows the use of 2′-deoxy uridines in primers for SNP assays to lower PCR product Tm.

FIG. 6 shows a comparison of standard barcode method vs UDG treatment method for hybridization of products to microarray.

FIG. 7 shows that increasing the number of 2′-deoxyuridines in the forward primer increases the signal and average pixel intensity

### DEFINITIONS

As used herein, the term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

As used herein, the term “sample” refers to anything capable of being analyzed by the methods provided herein that is suspected of containing a target nucleic acid sequence. Samples may be complex samples or mixed samples, which contain nucleic acids comprising multiple different nucleic acid sequences. Samples may comprise nucleic acids from more than one source (e.g. different species, different subspecies, etc.), subject, and/or individual. In some embodiments, the methods provided herein comprise purifying the sample or purifying the nucleic acid(s) from the sample. In some embodiments, the sample contains purified nucleic acid. In some embodiments, a sample is derived from a biological, clinical, environmental, research, forensic, or other source.

As used herein, the term “solid support” refers to any non-liquid support for performing a biological or other assay. Examples include, but are not limited to, arrays (e.g., microarrays), slides, particles, beads, etc.

As used herein, the term “barcode” or “DNA barcode” refers to a unique nucleic acid sequence (e.g., contained within a longer nucleic acid or oligonucleotide” that provides a unique identify to the nucleic acid. In some embodiments, a DNA barcode is one or more short gene sequences taken from a standardized portion of the genome that is used to identify species through reference to DNA sequence libraries or databases.

### DETAILED DESCRIPTION

Provided herein are methods for improving the detection sensitivity of amplification reaction products. In particular, provided herein are methods of improving sensitivity of detection of amplification products by introducing modified and degradable nucleotides into amplification primers.

For example, provided herein are systems and methods for improving signals obtained in biosensing applications (e.g., amplification reactions). In some embodiments, the present disclosure provides systems and methods for detection of amplification products on solid supports (e.g., microarrays) or in solution.

The systems and methods described herein improve signal by destruction of otherwise interfering competitive binding elements and improved hybridization through presentation of a single stranded element to a binding probe. For example, in some embodiments, one or more (e.g., 1, 2, 3, 4, 5, or all) of the thymidine nucleotides in one or more primer sequences is replaced by 2′-deoxyuridine. Following amplification, nucleic acids are treated with UDG (e.g., UDG) enzyme. The UDG enzyme transforms 2′-deoxyuridine base(s) into abasic nucleotides and upon heating the DNA is fragmented into smaller pieces. These smaller pieces do not bind as well to targets due to a significantly reduced Tm. Inclusion of multiple 2′-deoxyuridine nucleotides improves the signal further. Thus, unused primers in an amplification reaction are destroyed and do not bind to target probes in detection assays (e.g., microarrays). Destruction of the unincorporated primers solves the “dark primer problem” associated with microarrays as probes are often designed to capture targets that can overlap with the primer sequence.

In addition, primers that are incorporated into amplicons are cleaved from the double-stranded amplicon leaving a single stranded overhang. This single-stranded overhang is more amenable to binding with probe DNA and improves the hybridization and thus signal.

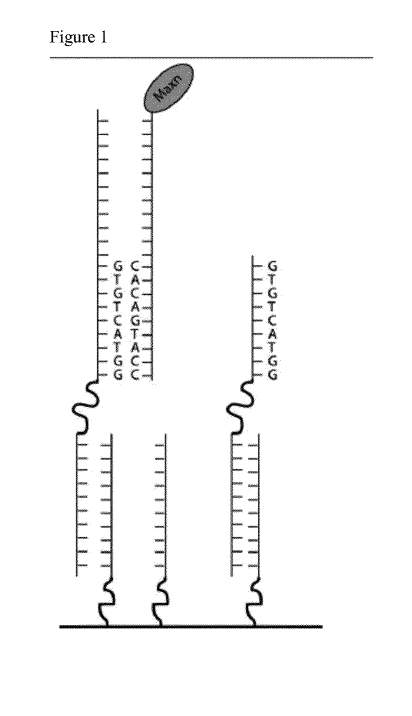

Prior to the present disclosure, the method described in FIG. 1 was often utilized. A forward primer with a unique barcode sequence attached by a linker is utilized. During PCR this forward primer is integrated into a double stranded PCR product with a label on the other strand. A hybridization array then captures the barcode sequence and the resulting PCR product (FIG. 1). The disadvantage of this process is that by not directly capturing the labeled DNA strand, there is competition for the capture reaction by unincorporated primer.

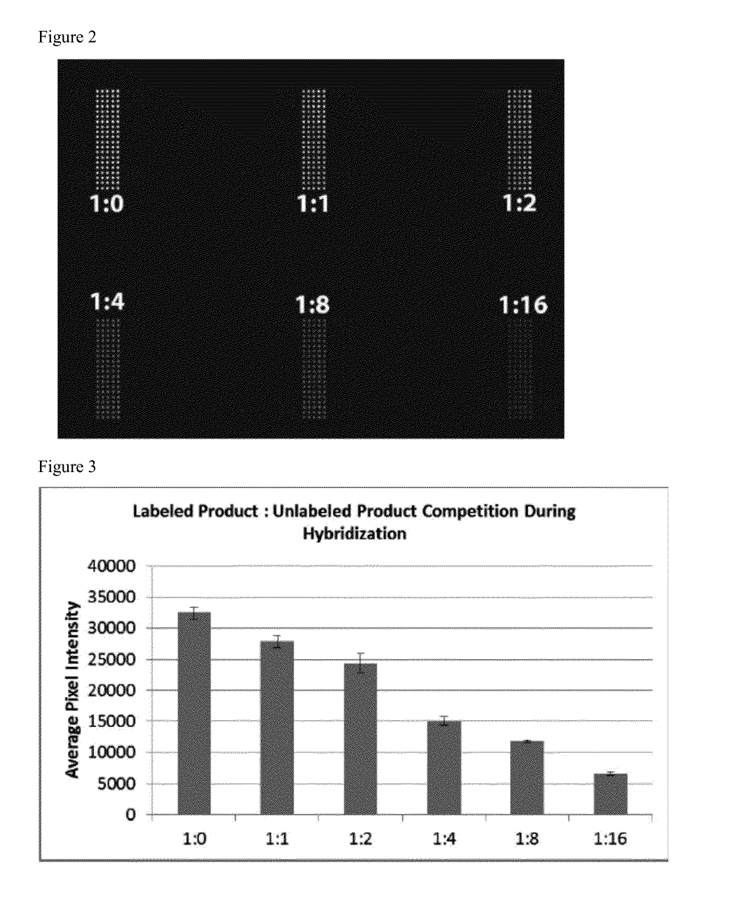

FIG. 2 shows how competition for unlabeled/unincorporated primer at the capture spot can affect hybridization efficiency for the target product. In this experiment unlabeled hybridization control was mixed at different ratios with labeled hybridization control. The complementary sequence was spotted onto a micro array and the hybridization reaction carried out (FIGS. 2 and 3). As the amount of unlabeled or competing probe increased the signal from the labeled probe decreased.

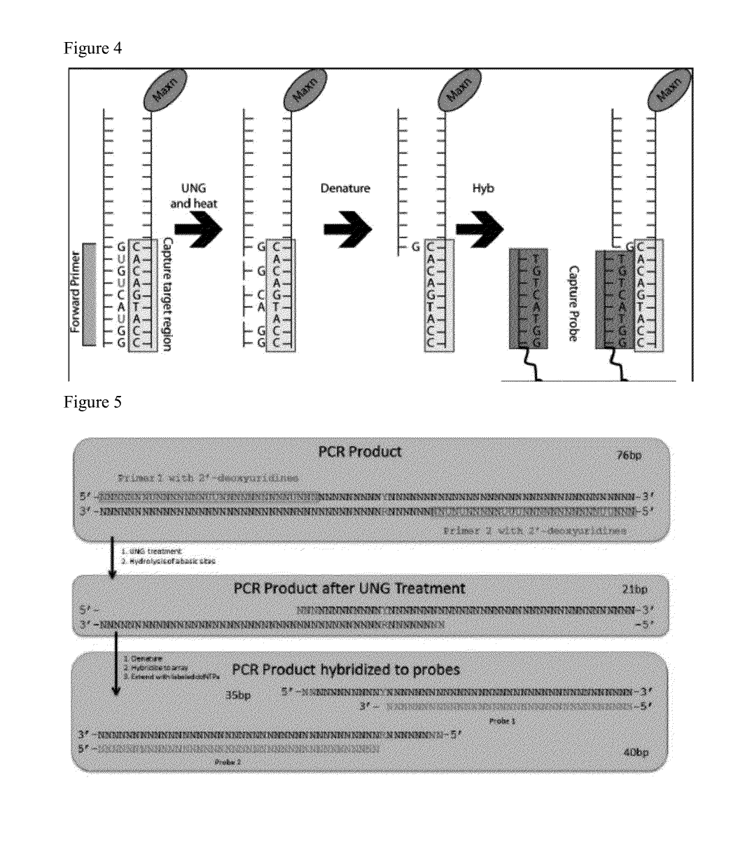

In contrast, embodiments of the present disclosure provide improved hybridization by incorporating degradable nucleotides into primers (e.g., 2′-deoxyuridines instead of thymidines or one or more ribonucleotides instead of deoxyribonucleotides) (FIG. 4). As PCR progresses double stranded products containing this forward primer accumulate. Following PCR, UDG enzyme and heat or other degradation methods are used to convert these 2′-deoxyuridines to abasic sites, which then degrade in the heat to cleave the DNA strand backbone. In addition, the UDG treatment degrades any remaining unincorporated forward primer ensuring it does not compete for binding during the array hybridization. This results in the forward primer disassociating from the PCR product at moderate temperatures freeing up the complimentary sequence on the fluorescently tagged strand for capture. In some embodiments, detection involves solution or array based capture with a capture sequence identical or similar to the forward primer sequence except thymidines are used instead of 2′-deoxyuridines. A benefit to this approach is that the DNA strand that has the fluorescent probe attached is directly probed. In some embodiments, the assay specificity is optimized by modifying amplification primers. Since the capture sequence is specific to DNA created by amplification, one can reduce the capture of dark or non-labeled DNA, which can result in bar coded tagged PCR primers as described above.

The systems and methods described herein further find use in polymorphism (e.g., SNP) detection assays. By incorporating 2′-deoxyuridines into the forward and reverse primers following UDG treatment the amplification duplex product that remains has as much lower TM, which facilitates denaturing of the product to allow for SNP probe hybridization. This is shown in FIG. 5. In some embodiments, rather than 2′-deoxyuridine based, RNA bases are placed in select positions of the PCR primer followed by RNAse treatment.

It is contemplated that the location(s) and amount of the 2′-deoxyuridines has an effect on results. For example, 2′-deoxyuridines located at the 5′ end of the primer do not lead to as much primer degradation as 2′-deoxyuridines in the central and 3′ end. A single 2′-deoxyuridine in the center of the primer result sin cleavage of the primer into two piece with one piece remaining attached to the duplex PCR product. A 2′-deoxyuridine in the center and one towards the 3′ results in the primer being cleaved in half along with being removed from the PCR product. The more 2′-deoxyuridines that are incorporated the more the primer will be degraded and the larger the reduction in Tm for the remaining fragments.

The present disclosure is not limited to PCR. Along with standard PCR, the incorporation of 2′-deoxyuridines also finds use in, for example, isothermal amplifications (e.g. Whole Genome Amplification, WGA/targeted WGA reactions) and other PCR methods (e.g. rolling circle, LAMP). Additionally, the incorporation of 2′-deoxyuridines finds use in Mass-spectrometry identification of PCR products by allowing the researcher to adjust the mass of potential products to increase the ability to distinguish between different products.

The present disclosure is not limited to array detection (e.g., optical microarray). Any suitable detection method finds use in the systems and methods described herein. Examples include, but are not limited to, detection of nucleic acids via colorimetric particle based methods and electrochemical methods (e.g., capture of DNA onto particles or surfaces followed by indirect detection via enzymatic conversion of redox reagents (e.g. similar to that used in hand-held cartridge systems such as i-STAT technologies (Abbott Laboratories, Abbott Park, Ill.)) or chemiluminesce methods (e.g. Luciferase type enzymes))).

Exemplary amplification and detection methods are described herein. In certain embodiments, the 2′-deoxyuridine based primer systems and methods described herein find use in amplification methods.

Exemplary amplification reactions include, but are not limited to the polymerase chain reaction (PCR), RT-PCR, or ligase chain reaction (LCR), each of which is driven by thermal cycling. Amplifications used in method or assays of the present disclosure may be performed in bulk and/or partitioned volumes (e.g. droplets). Alternative amplification reactions, which may be performed isothermally, also find use herein, such as branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), whole genome amplification, self-sustaining sequence replication, strand-displacement amplification, and the like.

Amplification may be performed with any suitable reagents (e.g. template nucleic acid (e.g. DNA or RNA), primers, probes, buffers, replication catalyzing enzyme (e.g. DNA polymerase, RNA polymerase), nucleotides, salts (e.g. MgCl₂), etc. In some embodiments, an amplification mixture includes any combination of at least one primer or primer pair, at least one probe, at least one replication enzyme (e.g., at least one polymerase, such as at least one DNA and/or RNA polymerase), and deoxynucleotide (and/or nucleotide) triphosphates (dNTPs and/or NTPs), etc.

In some embodiments, the present disclosure utilizes nucleic acid amplification that relies on alternating cycles of heating and cooling (i.e., thermal cycling) to achieve successive rounds of replication (e.g., PCR). In some embodiments, PCR is used to amplify target nucleic acids (e.g. partitioned targets). PCR may be performed by thermal cycling between two or more temperature set points, such as a higher melting (denaturation) temperature and a lower annealing/extension temperature, or among three or more temperature set points, such as a higher melting temperature, a lower annealing temperature, and an intermediate extension temperature, among others. PCR may be performed with a thermostable polymerase, such as Taq DNA polymerase (e.g., wild-type enzyme, a Stoffel fragment, FastStart polymerase, etc.), Pfu DNA polymerase, S-Tbr polymerase, Tth polymerase, Vent polymerase, or a combination thereof, among others. Typical PCR methods produce an exponential increase in the amount of a product amplicon over successive cycles, although linear PCR methods also find use in the present disclosure.

In some embodiments, the systems and methods described herein find use in sequencing methods (e.g., to identify DNA barcodes or other nucleic acid elements). Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

The methods, compositions, systems, and devices of the present disclosure make use of samples which include, or are suspected of including, a target nucleic acid sequence. Samples may be derived from any suitable source, and for purposes related to any field, including but not limited to diagnostics, research, forensics, epidemiology, pathology, archaeology, etc. A sample may be biological, environmental, forensic, veterinary, clinical, etc. in origin. Samples may include nucleic acid derived from any suitable source, including eukaryotes, prokaryotes (e.g. infectious bacteria), mammals, humans, non-human primates, canines, felines, bovines, equines, porcines, mice, viruses, etc. Samples may contain, e.g., whole organisms, organs, tissues, cells, organelles (e.g., chloroplasts, mitochondria), synthetic nucleic acid, cell lysate, etc. Nucleic acid present in a sample (e.g. target nucleic acid, template nucleic acid, non-target nucleic acid, contaminant nucleic acid may be of any type, e.g., genomic DNA, RNA, plasmids, bacteriophages, synthetic origin, natural origin, and/or artificial sequences (non-naturally occurring), synthetically-produced but naturally occurring sequences, etc. Biological specimens may, for example, include whole blood, lymphatic fluid, serum, plasma, sweat, tear, saliva, sputum, cerebrospinal (CSF) fluids, amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs or washes (e.g., oral, nasopharangeal, optic, rectal, intestinal, vaginal, epidermal, etc.) and/or other biological specimens.

In some embodiments, samples that find use with the present disclosure are mixed samples (e.g. containing mixed nucleic acid populations). In some embodiments, samples analyzed by methods herein contain, or may contain, a plurality of different nucleic acid sequences. In some embodiments, a sample (e.g. mixed sample) contains one or more nucleic acid molecules (e.g. 1 . . . 10 . . . 10² . . . 10³ . . . 10⁴ . . . 10⁵ . . . 10⁶ . . . 10⁷, etc.) that contain a target sequence of interest in a particular application. In some embodiments, a sample (e.g. mixed sample) contains zero nucleic acid molecules that contain a target sequence of interest in a particular application. In some embodiments, a sample (e.g. mixed sample) contains nucleic acid molecules with a plurality of different sequences that all contain a target sequence of interest. In some embodiments, a sample (e.g. mixed sample) contains one or more nucleic acid molecules (e.g. 1 . . . 10 . . . 10² . . . 10³ . . . 10⁴ . . . 10⁵ . . . 10⁶ . . . 10⁷, etc.) that do not contain a target sequence of interest in a particular application. In some embodiments, a sample (e.g. mixed sample) contains zero nucleic acid molecules that do not contain a target sequence of interest in a particular application. In some embodiments, a sample (e.g. mixed sample) contains nucleic acid molecules with a plurality of different sequences that do not contain a target sequence of interest. In some embodiments, a sample contains more nucleic acid molecules that do not contain a target sequence than nucleic acid molecules that do contain a target sequence (e.g. 1.01:1 . . . 2:1 . . . 5:1 . . . 10:1 . . . 20:1 . . . 50:1 . . . 10²:1 . . . 10³:1 . . . 10⁴:1 . . . 10⁵:1 . . . 10⁶:1 . . . 10⁷:1). In some embodiments, a sample contains more nucleic acid molecules that do contain a target sequence than nucleic acid molecules that do not contain a target sequence (e.g. 1.01:1 . . . 2:1 . . . 5:1 . . . 10:1 . . . 20:1 . . . 50:1 . . . 10²:1 . . . 10³:1 . . . 10⁴:1 . . . 10⁵:1 . . . 10⁶:1 . . . 10⁷:1). In some embodiments, a sample contains a single target sequence which may be present in one or more nucleic acid molecules in the sample. In some embodiments, a sample contains a two or more target sequences (e.g. 2, 3, 4, 5 . . . 10 . . . 20 . . . 50 . . . 100, etc.) which may each be present in one or more nucleic acid molecules in the sample.

In some embodiments, amplified target nucleic acids are identified using a nucleic acid probe specific for the target. In some embodiments, detection methods utilize capture nucleic acid probes on a solid support or in solution. In some embodiments, solid supports are microarrays. A DNA microarray, commonly known as gene chip, DNA chip, or biochip, is a collection of microscopic DNA spots attached to a solid surface (e.g., glass, plastic or silicon chip) forming an array for the purpose of detecting the presence of thousands of nucleic acids simultaneously. The affixed DNA segments are known as probes, thousands of which can be used in a single DNA microarray. Microarrays can be fabricated using a variety of technologies, including but not limiting: printing with fine-pointed pins onto glass slides; photolithography using pre-made masks; photolithography using dynamic micromirror devices; ink jet printing; or, electrochemistry on microelectrode arrays.

In some embodiments, amplified nucleic acids are captured in solution (e.g., on a bead or particle).

Captured or non-captured amplified nucleic acids are detected using any suitable method. In some embodiments, labeled probes are utilized. In some embodiments, detection is optical or electrochemical based. Other detection methods include, but are not limited to, detection of nucleic acids via colorimetric particle based methods and electrochemical methods (e.g., capture of DNA onto particles or surfaces followed by indirect detection via enzymatic conversion of redox reagents (e.g. hand held cartridges similar to that used in commercially available i-STAT technologies) or chemiluminescence methods (e.g. Luciferase type enzymes).

The systems and methods described herein find use in the detection of a variety of nucleic acid targets. Examples include, but are not limited to pathogens (e.g., pathogenic microorganisms such as viruses, bacteria, and single cell eukaryotic pathogens (e.g., fUDGi or parasites), polymorphisms (e.g., SNPs), and other variant nucleic acids (e.g., mutants, copy number variations, genomic deletions and duplications and the like).

In some embodiments, compositions, kits and systems are provided. In some embodiments, compositions, kits, or systems comprise probes, amplification oligonucleotides comprising 2′-deoxyuridine nucleotides, polymerases, nucleotide, detection and/or capture probes and the like. In some embodiments, kits include all components necessary, sufficient or useful for amplifying and detecting target nucleic acids (e.g., reagents, controls, instructions, etc.). The kits described herein find use in research, therapeutic, screening, and clinical applications.

Embodiments of the present invention provide complexes of target nucleic acids and one or more amplification primers comprising 2′-deoxyuridine nucleotides.

### EXAMPLES

### Example 1

### Methods

Primers targeting Bacillus anthracis were ordered with 2′-deoxyuridines instead of thyamines from IDT.

The following sequences were used (Table 1).

TABLE 1

| | | # of |
|---|---|---|
| Primer | Sequence 5;→3′ | Uracils |
| infB- | 5′-GUG GUG CAC AAG UAA CGG AUA UUA | 7 |
| NF3dU | CAA UCA | |
| sspe- | 5′-AAA CAA GCA AAC GCA CAA UCA GAA | 1 |
| NF3dU | GCT | |
| pxo1- | 5′-ACA CAU UAC UGA UGG UUU UGA UUU | 14 |
| NF3-dU | CUU AGG CUT | |
| pxo2- | 5′-GUG UAA AAG GUA AAA AAU GGA GUC | |
| NF3dU | CGA UUA AGG | |

The reverse primers (Table 2) were labeled with a fluorescent dye (Max dye from IDT) for detection on the hybridization array.

TABLE 2

| | |
|---|---|
| BA-INFB-R | /5MAXN/CTCCTGCTGCTTTCGCATGGTTAATC |
| BA-SSPE-R | /5MAXN/TCTGTACCATAACTAGCATTTGTGCTTTGAA |
| BA-PXO1-R | /5MAXN/TGACACTTTGGTTGGATGGTTTAATGAAGAG |
| BA-PXO2-R | /5MAXN/TGGAGATTCTTCAACGCAATATACCCTACTAAA |

PCR mastermix was made with the above primers and included the following (Table 3):

Base reagent mix

| | | | | total uL to add |
|---|---|---|---|---|
| Reagent | Initial conc | Final conc | uL to add | for master mix |
| Kappa buffer A | 5x | 1x | 60 | 60 |
| MgCl2 | 25 mM | 3.5 mM | 42 | 42 |
| | | additional | | |
| dNTPs | 10 mM | 0.2 mM | 6 | 6 |
| Polymerase | | 0.4 uL/50 uL | 2.4 | 2.4 |

| | | |
|---|---|---|
| total | 110.4 | 110.4 |

1:10 dilutions of 500 μM primer stocks were made (Table 4)

Partial plex n = 1

| | Stock conc | Final conc | |
|---|---|---|---|
| Base reagent mix | 1x | 1x | 110.4 |
| Primer 1F (infB) | 50 uM | 0.6 uM | 3.6 |
| Primer 1R | 50 uM | 0.6 uM | 3.6 |
| (infB) | | | |
| Primer 2F | 50 uM | 0.2 uM | 1.2 |
| (sspE) | | | |
| Primer 2R | 50 uM | 0.2 uM | 1.2 |
| (sspE) | | | |
| Primer 3F | 50 uM | 0.2 uM | 1.2 |
| (pxo1) | | | |
| Primer 3R | 50 uM | 0.2 uM | 1.2 |
| (pxo1) | | | |
| Primer 4F | 50 uM | 0.2 uM | 1.2 |
| (pxo2) | | | |
| Primer 4R | 50 uM | 0.2 uM | 1.2 |
| (pxo2) | | | |
| DNA | | | 2.5 |
| Antifoam A | 2% | | 6 |
| Water | | | 166.7 |
| total | | | 300 |

Bacillus anthracis Sterne strain DNA (Template concentration ˜1 ng/μL) was spiked into the reaction and PCR was performed. Following PCR, UDG was added to each PCR reaction and the reactions incubated for 10 min at 37° C. to convert the 2′-deoxyuridine sites in the forward primer to an abasic site. The PCR reaction was then heated to 95° C. for 2 min to denature and degrade the abasic sites. As a control no UDG was added to one reaction.

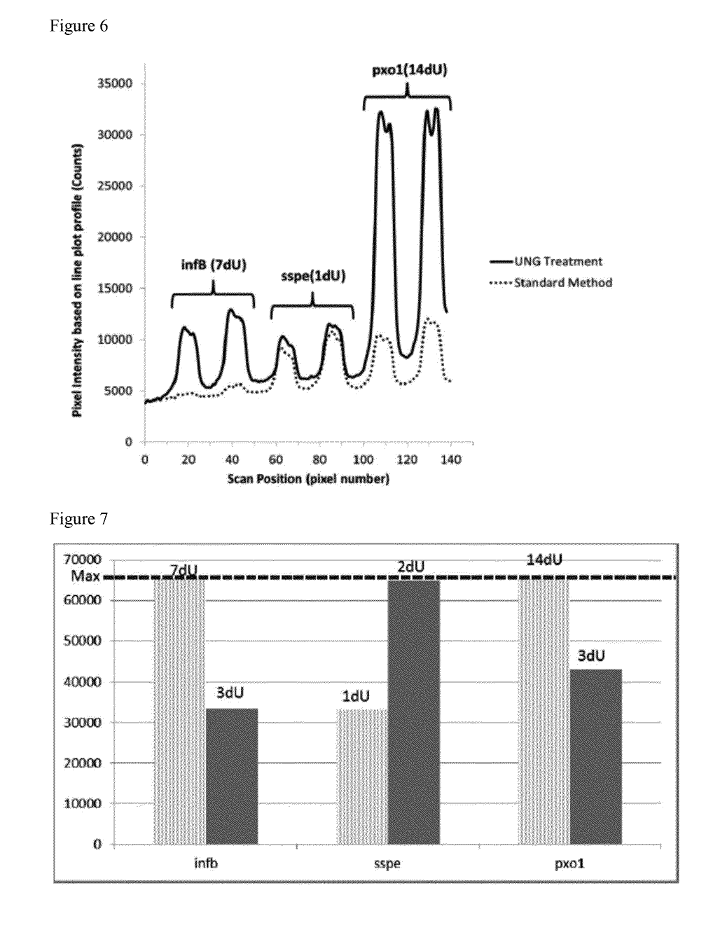

An array was made with capture sequences identical to the forward primer sequence except thyamines were used instead of 2′-deoxyuridines. However, these probes can also contain any of the standard spacer elements often used (e.g. 20T, C6, C12, PEG, etc. . . . ) Each capture sequence was spotted down in duplicate on the array. The UDG treated PCR reaction and standard PCR reaction were incubated on the slide at 50° C. for 10 min followed by three washes with 0.2×SSPE (0.2×SSPE consists of 0.002 M phosphate buffer, 0.0298 M NaCl, 0.0002 M EDTA at a pH of 7.4). Arrays were then imaged and spot intensities examined for each of the 3 target loci tested. Line plots were generated through the array spots to assess the performance of each primer set (FIG. 6 and Table 5).

TABLE 5

| | | Number of | Signal Ratio (Ung/ |
|---|---|---|---|
| Peak | Target loci | Uracils | Std) |
| 1 | infB | 7 | 13.67 |
| 2 | infB | 7 | 6.62 |
| 3 | sspe | 1 | 1.11 |
| 4 | sspe | 1 | 1.16 |
| 5 | pxo1 | 14 | 5.53 |
| 6a | pxo1 | 14 | 4.02 |

As shown in Table 5, incorporation of 2′-deoxyuridines into the primer and treatment with UDG following PCR improved signal in the array. The more 2′-deoxyuridines the primer contained, the more it was degraded during UDG treatment ensuring the capture site on the amplified nucleic acid target was exposed for the capture sequence. Table 5 shows the peak pixel intensity between the UDG treated and control reactions. It is demonstrated that having multiple 2′-deoxyuridines in the primer provides a better signal increase due to breaking the incorporated primer into smaller pieces that cannot rehybridize to the PCR product at hybridization temperatures. The incorporation of 2′-deoxyuridines improved the signal ˜14-fold for one target and 5 fold for another target. This incorporation of 2′-deoxyuridines improves improve sensitivity, selectivity, and specificity of targets.

In the data shown in FIG. 5, neither primer of the primer pairs employed contains a detectable label, but both contain deoxy 2′-deoxy uridines. Detection is accomplished after hybridization of the PCR product after treatment with UNG (UDG) to a probe attached to a solid support via its 5′ terminus. Incorporation of a single chain-terminating nucleotide (containing a detectable label) complementary to the first nucleotide at its 3′ terminus is one method that enables the detection of a particular SNP. Fluorescently labeled dideoxy nucleotide triphosphates are one example of the chain-terminating nucleotide triphosphates that could be used. FIG. 7 demonstrated that increasing the number of 2′-deoxyuridines used in the forward primer increases the signal when using this hybridization strategy.

Although the disclosure has been described in connection with specific preferred embodiments, it should be understood that the disclosure as claimed should not be unduly limited to such specific embodiments. Various modification and variation of the described methods and compositions of the disclosure will be apparent to those skilled in the art without departing from the scope and spirit of the disclosure. Indeed, various modifications of the described modes for carrying out the disclosure understood by those skilled in the relevant fields are intended to be within the scope of the following claims. All publications and patents mentioned in the present application are herein incorporated by reference.

We claim:

1. A system, comprising:
a) a plurality of amplification primer pairs specific for a target nucleic acid, wherein at least one thymidine nucleotide in one of said primers of each pair is replaced with 2′-deoxyuridine and the other primer of each of said pairs comprises a detectable label; and
b) a detection device.

2. The system of claim 1, wherein said detection device is an optical, colorimetric, chemiluminescent, electrical, radio isotope, chemical change, magnetic, or electrochemical detection device.

3. The system of claim 1, wherein said system further comprises a solid support comprising a plurality of nucleic acids complementary to said target nucleic acids.

4. The system of claim 3, wherein said solid support is a microarray.

5. The system of claim 3, wherein said nucleic acids complementary to said target nucleic acids comprise one or more of a modified nucleic acid base, a detectable label, or a quencher.

6. The system of claim 1, wherein said primer pairs are selected from PCR primers, isothermal amplification primers, and whole genome amplification primers.

7. The system of claim 1, wherein at least two of said thymidine nucleotide in one of said primers of each pair is replaced with 2′-deoxyuridine.

8. The system of claim 1, wherein all of said thymidine nucleotide in one of said primers of each pair are replaced with 2′-deoxyuridine.

9. The system of claim 1, wherein said amplification primers are selected from primers for PCR, RT-PCR, whole genome amplification, isothermal amplification, and rolling circle replication.

10. The system of claim 1, wherein said system further comprises a UDG enzyme.

11. A method, comprising:
a) contacting a sample comprising a target nucleic acid with a plurality of amplification primer pairs specific for a target nucleic acid, wherein at least one thymidine nucleotide in one or more of said primers of each pair is replaced with 2′-deoxyuridine and the other primer of each of said pairs comprises a detectable label;
b) performing an amplification reaction to amplify said target nucleic acid;
c) treating said amplification reaction with a UDG enzyme; and
d) capturing said target nucleic acid on a solid support comprising a plurality of nucleic acids complementary to said target nucleic acids.

12. The method of claim 11, further comprising the step of detecting said target nucleic acid using a detection device.

13. The method of claim 12, wherein said detection device is wherein said detection device is an optical, colorimetric, chemiluminescent, electrical, radio isotope, chemical change, magnetic, or electrochemical detection device.

14. The method of claim 11, wherein said solid support is a microarray.

15. The method of claim 11, wherein said nucleic acids complementary to said target nucleic acids comprise one or more of a modified nucleic acid base, a detectable label, or a quencher.

16. The method of claim 11, wherein said primer pairs are selected from PCR primers, isothermal amplification primers, and whole genome amplification primers.

17. The method of claim 11, wherein at least two of said thymidine nucleotide in one of said primers of each pair is replaced with 2′-deoxyuridine.

18. The method of claim 11, wherein all of said thymidine nucleotide in one of said primers of each pair are replaced with 2′-deoxyuridine.

19. The method of claim 11, wherein said amplification is selected from PCR, RT-PCR, whole genome amplification, isothermal amplification, and rolling circle replication.

20. A reaction mixture comprising at least one complex of a target nucleic acid and one or more amplification primers, wherein at least one thymidine nucleotide in one or more of said primers is replaced with 2′-deoxyuridine.
